(19) United States
(12) Patent Application Publication
(10) Pub. No.: US 2023/0197266 A1
(43) Pub. Date: Jun. 22, 2023

(54) ELECTRONIC PERSONAL PROTECTIVE DEVICE

(71) Applicants: Marco Mario FAITA, Lissone (Monza Brianza) (IT); MARTELLA S.R.L., Milano (IT)

(72) Inventors: Marco Mario FAITA, Lissone (Monza Brianza) (IT); Simona MARTELLA, Legnano (Milano (IT)

(21) Appl. No.: 17/998,843

(22) PCT Filed: May 14, 2021
(86) PCT No.: PCT/IB2021/054147

§ 371 Date: Nov. 15, 2022

(30) Foreign Application Priority Data
May 15, 2020 (IT) 102020000011311

(51) Int. Cl. G16H 40/67; G01S 17/74; G01S 13/76; G01S 13/88; G01S 13/86; G08B 21/02

(52) U.S. Cl. CPC G16H 40/67; G01S 17/74; G01S 13/765; G01S 13/886; G01S 13/865; G08B 21/02

(57) ABSTRACT

The present invention describes an electronic personal protective device (2) comprising a rechargeable battery (3), a transceiver (4) configured to send and receive wireless signals (FR1, FR2) with predetermined power and sensitivity as a function of the action radius (r) which is to be monitored, a memory unit (5) containing a unique recognition code (ID1, ID2), an actuator (6) configured to warn the person wearing said electronic device (2), a processing unit (20) configured to: generate and send to said transceiver (4) a frame (FR1, FR2) at programmable time intervals (Tadv), receive and process frames (FR1, FR2) sent from other electronic personal protective devices (2) placed within said action radius (r), generate an alarm signal (S_AL), if a second electronic device (2) is located within said action radius (r), send said alarm signal (S_AL) to said actuator (6). The invention further describes a corresponding method and computer program.

### TECHNICAL FIELD

The present invention relates to an electronic personal protective device that allows social distancing among people, tracking the contacts among people and measuring the time of use of Personal Protective Equipment (“PPE”) of a known type.

In particular, the present invention relates to a system.

In the following discussion, the electronic personal protective device according to the present invention is also referred to with the term “CoviTag®”, “infra-red beacon” or “radio beacon”.

Furthermore, by “radio beacon” in the following discussion it is meant an omnidirectional radio transmitter that continuously transmits a signal on a specific frequency.

### PRIOR ART

Worldwide, governments and health authorities are working together to find solutions to the COVID-19 pandemic, to protect people and to get society back to normal. In this context, many health authorities and major IT players are investing huge resources to create specific smartphone applications to be used for tracking people.

Applications on smartphones that are able to track, using GPS, the paths travelled by the smartphone owner and to possibly calculate the distances between people using Bluetooth technology are known.

Sw applications that use Background Geolocation but only work outdoors and not indoors, such as inside buildings, are currently being developed.

However, using these applications involves high battery consumption.

Furthermore, the traceability is not precise, as when these applications work in the background, the application cannot make “announcements” and “discoveries” (advertising) and has limitations for example for the random reading of IDs for privacy reasons. In fact, many smartphone applications currently used must necessarily always be active with the screen always highlighted.

When calculating the distance between people, such smartphone applications tend to collapse easily if there are too many devices in the action radius.

One of the objects of the invention described herein is to warn people that the distance with respect to another person is less than a predetermined or pre-programmed value or threshold.

In other words, the object of the present invention is to measure the distance between two people.

A further object of the present invention is to monitor the maximum residence and exposure time.

Another object of the present invention is to allow the electronic device to be activated on a specific ID.

A further object of the present invention is to warn the user that their PPE is close to expiry and needs to be replaced.

Another object of the present invention is to determine the traceability of the person's contacts.

A further object of the present invention is to improve the safety level of people.

Another object of the present invention is to provide an electronic personal protective device which is reliable and efficient in its use.

### OBJECT OF THE INVENTION

In a first aspect of the invention, the above-mentioned objects are achieved by an electronic personal protective device according to what is disclosed in claim 1.

Advantageous aspects are described in dependent claims 2 to 13.

In a second aspect of the invention, the above-mentioned objects are achieved by an electronic personal protective device, in particular a facial protection mask according to what is disclosed in claim 14.

Advantageous aspects are described in dependent claim 15.

In a third aspect of the invention, the above-mentioned objects are achieved by a safety system for tracking movements and respect for distances between people, according to what is disclosed in claim 16.

Advantageous aspects are described in dependent claims 17 and 18.

In a fourth aspect of the invention. the above-mentioned objects are achieved by a safety method for tracking movements and respect for distances between people according to what is disclosed in claim 19.

Advantageous aspects are described in dependent claim 20.

In a fifth aspect, the invention describes a computer program, which when running on a computer implements at least one or more steps of the method according to the second aspect of the invention, according to what is disclosed in claim 21.

In general, the invention offers the following technical effects:
allows an increase in the safety of people or users in places such as is hospitals, factories, warehouses, plants, offices, points of sale, public transport and the like;
allows checking the distancing between the people wearing it;
allows warning people who get too close about the possible violation of the rules on social distancing;
allows tracking people who pass by certain points of interest;
allows tracking any violations of the safety distances;
allows reducing battery consumption, both of the device and of the smartphone;
allows not to interfere with other electronic devices, such as electro-medical equipment;
allows the device to work even in the presence of high solar energy;
allows the device to work even in the presence of obstacles and barriers, such as walls;
allows avoiding false positive reports of devices positioned outside the line of sight, for example placed behind walls or separations;
allows the device to work independently, therefore with no need to rely on a fixed coordination and control infrastructure.

The technical effects/advantages mentioned, and other technical effects/advantages of the invention, will emerge in further detail from the description provided herein below of an example embodiment provided by way of approximate and non-limiting example with reference to the appended drawings.

### BRIEF DESCRIPTION OF THE DRAWINGS

For a better understanding of the invention and to appreciate the advantages thereof, some non-limiting example embodiments thereof are described herein below, referring to the appended drawings, in which:

FIG. 1 illustrates an example of the system of the invention adapted to maintain the safety distances between people in a top view;

FIG. 2 illustrates an electronic device of FIG. 1, in side view;

FIG. 3 illustrates the action radius of each device of FIG. 1;

FIG. 4 illustrates a block diagram of the system of FIG. 1 according to a first embodiment according to the invention;

FIG. 5 illustrates a personal protective device worn by a person and comprising an electronic device;

FIG. 6 illustrates a detail of the lens of an infra-red transceiver of the electronic personal protective device;

FIG. 7 illustrates an example in which some electronic devices are fixed in certain points of passage;

FIG. 8 illustrates a block diagram of the infra-red transceiver;

FIG. 9 schematically illustrates the processing of the received infra-red signal IR-TX, which is output as waveform IR-FRAME;

FIG. 10 schematically illustrates the use of a carrier for filtering any interference due to sunlight or ambient light;

FIG. 11 illustrates the physical layer of the infra-red communication protocol according to the invention;

FIG. 12 illustrates an anti-collision protocol in the presence of two or more electronic personal protective devices;

FIG. 13 schematically illustrates the trend of the sensitivity of an infra-red transceiver to ambient light;

FIG. 14 schematically illustrates a configuration of the reception LEDs and two superimposed multi-axis infra-red emission lobes;

FIGS. 15 and 16 illustrate the configuration for mounting the infra-red LEDs of FIG. 14 on printed circuit boards (PCBs);

FIG. 17 schematically illustrates the calculation of the distance d between two electronic devices with infra-red transceivers;

FIG. 18 illustrates an embodiment of the processing unit according to the present invention;

FIG. 19 illustrates a preferred embodiment of the electronic personal protective device;

FIG. 20 illustrates a preferred embodiment of the electronic personal protective device;

FIG. 21 illustrates a preferred embodiment of the electronic personal protective device;

FIG. 22 illustrates a preferred embodiment of the electronic personal protective device;

FIG. 23 illustrates a preferred embodiment of the electronic personal protective device;

FIGS. 24 and 25 illustrate two characteristic curves.

### DETAILED DESCRIPTION OF PREFERRED EMBODIMENTS OF THE INVENTION

It should be observed that in the following description, identical or analogous blocks, components or modules are indicated in the figures with the same numerical references, even though they are illustrated in different embodiments of the invention.

With reference to the cited figures, the safety system for tracking movements and respect for distances between people according to the invention is indicated overall with the reference number 1 in the block diagram of FIG. 1, 3 or 4.

The system comprises a first electronic personal protective device 2 worn by a person and at least one second electronic personal protective device 2 worn by a second person.

In FIGS. 1, 3 and 4 the system 1 is illustrated for simplicity of exposure, with the presence of only two electronic personal protective devices, each worn by a person.

The electronic device 2 comprises a box-shaped casing shaped to be worn by a person in the form of a button (round or square or rectangular), pin, bracelet (wearable on the wrist), pendant to be applied to a necklace, badge strap, belt or armband.

The electronic device 2 can also be applied to a personal protective device, in particular facial protection mask 60, illustrated in FIG. 5.

In a first aspect, the present invention relates to an electronic personal protective device 2 comprising a rechargeable battery (3) a transceiver 4 configured to send and receive wireless signals (FR1, FR2); a memory unit (5) containing a unique recognition code (ID1, ID2); an actuator (6) configured to warn the person wearing said electronic device 2; and a processing unit 20.

Preferably, the battery is a rechargeable lithium battery.

The battery 3 can be recharged either wirelessly or by connecting it via cable to an external charger.

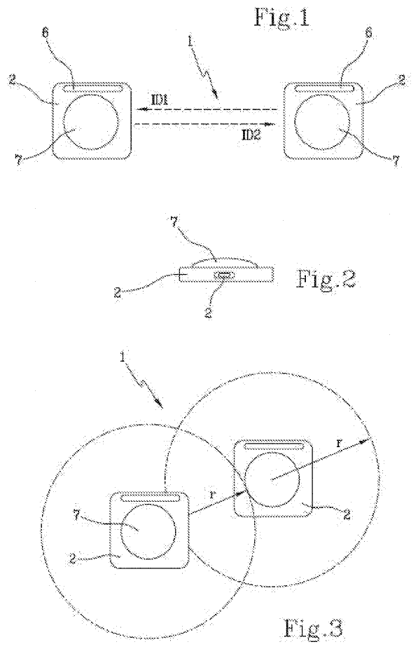

In this case, the coviTAG 2 is provided with a USB port 8, accessible from the outside of the casing of the coviTAG 2, shown in FIG. 2.

Preferably, the memory unit 5 is of the E2PROM, RAM, FLASH type and contains at least one unique and unrepeatable ID, for example, 96-bit.

The actuator 6 is configured to warn and signal the person wearing the electronic device 2 by means of a light signal (for example, by using high-luminosity RGB LEDs 6b) and/or a vibro-acoustic warning device (Buzzer 6a) on certain events, such as, for example, the safety distance has been exceeded, the level of battery charge, the approach of the expiry date of the PPE, any malfunctions.

The unique ID code can be stored during production step or when the electronic device 2 is associated with the person who will wear it. Once worn, the electronic device 2 stores the unique ID code of all the identical devices 2 it encounters and which are at a predetermined and programmable distance, by way of a non-limiting example, shorter than one meter.

All the IDs encountered are accumulated and stored in the memory unit 5 of the CoviTAG 2.

This function allows the backward traceability of the devices and therefore of the people who wore them in order to identify the movements or any contagion paths.

Preferably the CoviTAG 2 comprises a timer configured to send a signal to the actuator (6) able to warn the user wearing the personal protective device of the approach of the expiry date.

In this case, the actuator 6 of the device 2 keeps a green LED active for the entire period of validity of the PPE, and it turns red or off when the device 2 associated with the monitored object exceeds the programmed expiry time.

The association CoviTAG 2—person who will wear it is made through appropriate software preferably installed on a PC or APP from a smartphone/Tablet. Within any organization, the people in charge, before distributing the coviTAGs 2 to the staff, must connect them via USB or BT BLE with a PC running the software.

At this point, the operator will assign the coviTAG 2 simply by loading a list of names previously loaded in an excel® type spreadsheet, the software will automatically open the spreadsheet in order to read the unique ID code present in the CoviTAG 2, and pair PersonName—ID coviTAG 2. The pairing thus made is loaded into a file and stored in the PC in encrypted mode, accessible only with a password. Once the pairing has been made, the coviTAG can be delivered to the person, upon completing the procedure it will be possible to proceed with writing the name of the person assigned in the space provided (on the bottom) of the coviTAG arranged to receive a label. The coviTAG also has a USB socket to be used for charging the LiPo type battery. The coviTAG 2 warns the user of the approach of low battery by setting a specific warning colour on the RGB LED present on the coviTAG in order to draw the attention.

The USB socket 8 is configured to recharge said battery 3 and/or to programme and configure the processing unit 20.

The processing unit 20 configured to generate and send to said transceiver 4 a frame (FR1, FR2) at programmable time intervals (Tadv); to receive and process frames (FR1, FR2) sent from other electronic personal protective devices 2 placed within said action radio (r); to generate an alarm signal (S_AL), if a second electronic device 2 is located within said action radius (r); to send said alarm signal (S_AL) to said actuator (6).

In general, it should be noted that in the present context and in the subsequent claims, the processing unit 20 is considered to be divided into distinct functional modules (memory modules or operating modules) for the sole purpose of describing the functionalities thereof clearly and completely.

Such processing unit can consist of a single electronic device, appropriately programmed to perform the functionalities described, and the different modules can correspond to hardware entities and/or routine software that are part of the programmed device.

Alternatively or additionally, these functionalities can be executed by a plurality of electronic devices on which the aforesaid functional modules can be distributed.

The processing unit 20 can further make use of one or more processors for executing the instructions contained in the memory modules.

In a preferred embodiment, the transceiver 4 is an infra-red (IR) transceiver 4, configured to transmit and receive said wireless signals in the infra-red spectrum.

Devices 2 with transceiver 4 exclusively with infra-red rays IR have the advantage of low production cost, very low consumption, do not require any authorisation from authorities in order to operate, physical confinement of the signal (the IR signal cannot pass through walls, obstacles or leave a room). They can therefore be used in environments where radio waves are not allowed.

Using a specific infra-red transceiver 4, placed behind a Fresnel lens 7, an IR channel is obtained (infra-red-beacon).

Each device 2 continuously sends, outside the lens 7, at programmed time intervals, an IR signal according to a protocol called “IRBEACON”, described further on.

By adjusting the emission power of the IR LED present in the transceiver 4 it is possible to adjust the action radius r and therefore the detectable distance between two or more devices 2.

Each coviTAG 2 continuously emits a sequence of codes or frames FR1, FR2 according to the IRBEACON protocol on the infra-red channel. The emission power is programmed to cover only the desired safety distance r. The infra-red receiver 4 of the coviTAG 2 is provided with suitable circuits to adapt its sensitivity as a function of the detection distance.

The immunity to background (environmental) IR radiation is guaranteed with the use of receivers equipped with an automatic gain control system (AGC).

The IRBEACON protocol allows the management of several coviTAGs 2 present in the same area being based on techniques borrowed from algorithms of the CSMA/CD or “Hola” type (hola.org).

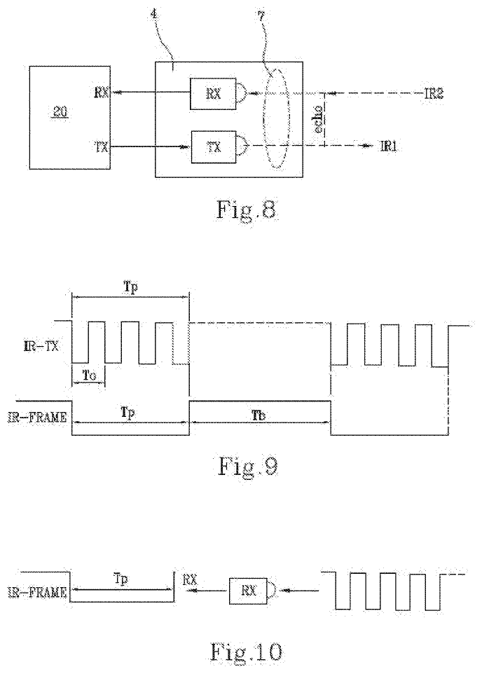

FIG. 8 illustrates the block diagram of the infra-red transceiver 4.

The IRBEACON protocol comprises a physical layer with a Pulse Code Modulation (PCM) with a carrier comprised between 10 KHz and 600 KHz, preferably, comprised between 30 KHz and 200 KHz.

The pulse code modulation is shown in FIGS. 9 and 10, where [To]=Carrier frequency of the transmitted IR signal (preferably, 30-200 kHz), [Tp]=IR pulse duration from 30 to 800 us, advantageously from 50 at 500 uS, [Tb]=pause time between two pulses.

The transmitted infra-red signal IR-TX is received by the IR receiver and processed in such a way as to output the IR-FRAME waveform.

The use of a carrier Fo=1/To gives greater filter capacity and immunity to background IR radiation from sunlight interference and any lamps powered at 50 Hz. The output of the Receiver 4 is further stabilised using signal amplification circuits such as Automatic Gain Controller (AGC).

Frame Format

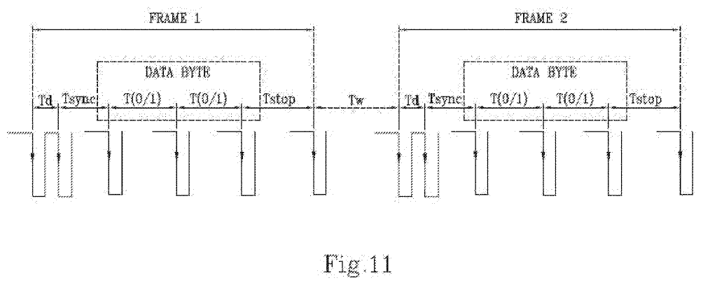

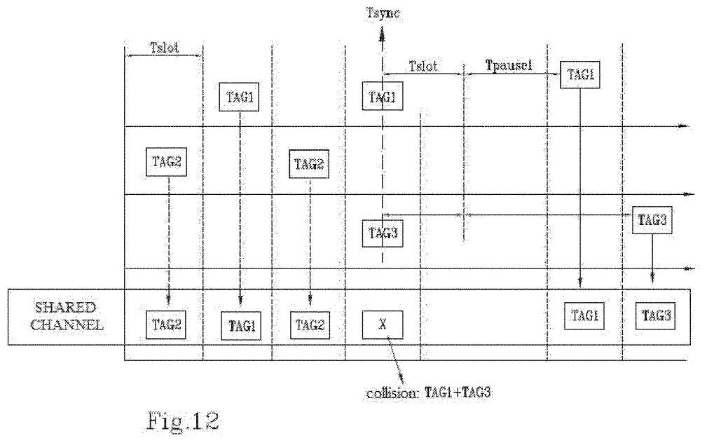

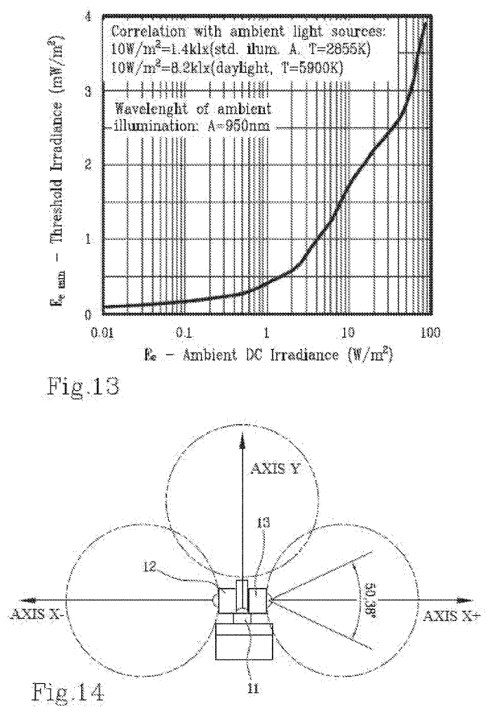

FIG. 11 illustrates the format of the frames FR1 and FR2.

Table 1 reports a possible time composition of the IR signal as illustrated in FIGS. 9, 10 and 11.

Table 2 illustrates the sequence of byte packets that makes up a frame.

Table 3 shows the minimum and maximum duration of a Frame in the hypothesis that all bits=0 or all bits=1 are sent and are the two extreme cases.

Table 4 indicates that there is always a pause time Tw between two frames.

Between Frame1 and Frame2 of Table 1, there is a Pause time Tw.

Example of byte sending: 11 00 11 01→TdTd . . . Td Tsync T(0/1) T(0/1) . . . T(0/1) Tstop.

IRBEACON Protocol

The IRBEACON protocol for transmitting infra-red frames according to the present invention comprises one or more of the following steps:
<AGCSTART><UUID><MAJOR><MINOR><TXPOWER><CRC>
1. AGCSTART: sending a sequence of (n>1) dummy pulses of duration [T] used to couple/stabilise the AGC of each receiver.
2. UUID: composed of 16 bytes, 10-bytes for the name space and 6-bytes for the instance. The first is intended to ensure a unique ID across multiple platforms. It is used to filter scanned beacons.
3. Major: 2 bytes that group a relative set of Beacons; (grouping by classes).
4. Minor: 2 bytes unique identifier of a specific beacon;
5. TxPower: 1 byte RSSI value expressed in [dB].
6. CRC: 2 bytes (16 bits) with any standard polynomial calculation technique from UUID-TXPOWER.

TABLE 5

| | STORE/LOCATION | | |
|---|---|---|---|
| | MILAN | PARIS | LONDON |
| UUID | F9B9EC1C-3925-53C3-90A9-1E39D4CEB77F | | |
| MAJOR | 1 | 2 | 3 |
| MINOR | 10 | 10 | 10 |
| | 20 | 20 | 20 |
| | 30 | 30 | 30 |

Example of Possible Coding

The unique identification code ID or UUID is shared by all the stores of the same chain of department stores or indicates a mask manufacturer or a company that purchased them. This allows the devices in use to uniquely identify each of them in a single region/place. Following the example, for each of them: San Francisco, Paris and London are assigned a unique value (Major) that distinguishes it from the other one. Within each single store, there are beacons with ID or UUID and Major codes assigned as described, but the Minor value is different for each department. In this way a smartphone by reading this grouping is able to know where it is and allow relative notifications to be sent in the department of competence.

Infrared Multi-Beacon Detection

With reference to the IRBECON protocol, the present protocol processing step is used to establish the presence of one or more beacons with a distance shorter than that allowed and the exchange and recognition of one's own ID.

Each infra-red beacon constantly keeps the RX channel being received, at programmable time intervals—advertising time—Ta comprised between 100-500 mS—sends its own FRAME according to the above described IRBEACON protocol. If a coviTAG 2 is present in the action radius of the sent IR pulse, the coviTAG 2 once it has received the FRAME will understand that it is at a distance that allows detection and will therefore activate the proximity alarm (activation of LED 6b or Buzzer 6a).

The IR emission power and the detection sensitivity of the receiver 4 are two parameters that are suitably designed to guarantee detection according to an action radius or range r, for example, with r equal to a distance of less than 1 m or 2 m (distances imposed in the covid-19 era).

IRBEACON Collision Detection

A certain number of coviTAGs 2 can be simultaneously active and present within the action radius of the IR signal. This means that the transmission [IR] channel comprised between 20-200 kHz can be occupied by any other coviTAG. By occupied it is meant the concomitant superposition (collision) of several [IR] beams sent simultaneously with the same carrier at the same instant of time.

The final effect of this superposition is the immediate destruction of any coding and the impossibility for the CPU to retrieve the ID according to the IRBEACON protocol.

Collisions reduce the speed of data collection, increase the delay in identification and degrade the efficiency and reliability of the transmission system. The collision of the infra-red rays transmitted by the various coviTAGs 2 is inevitable due to the non-cooperation mechanism between the coviTAGs 2, each electronic device 2 is asynchronous with respect to the others.

Therefore, an anti-collision method of the signals transmitted by the coviTAGs 2 is a key factor that affects the identification efficiency.

To overcome this problem, the protocol is completed with the following collision detection protocol.

Each CoviTAG 2 monitors the infra-red reception channel [Rx] at pre-established time intervals [Ts], and carries out the following steps.

This configuration allows detecting the echo of the sent FR frame (illustrated in FIG. 8).

With reference to FIG. 12, the IRBEACOM protocol contemplates the following operating steps for the various devices coviTAG 2:

1. Each device coviTAG 2 listens to the IR channel (SHARED CHANNEL), if it does not detect the start of other transmissions, it switches to step 2. Otherwise, if “Starting transmission of IR-FRAME transmitted=echo FRAME received?”: It means that there are no other coviTAG 2 in the action radius.

2. IR-FRAME transmitted < > echo FRAME received (COLLISION): presence of other beacons within the action radius.
a. All coviTAGs that at time [Tysnc] detect the collision, immediately interrupt the transmission, pause for a time Tpause calculated as a number comprised between 0 and 2\hat{ }(K−1)*T, where T is the time of transmission of the message and [K] is a coefficient that takes into account the number of collisions that have already occurred.
b. The TAGs that detect the collision resume listening on the IR channel, always waiting at least for a time [Tslot] calculated starting from time [Tsync]+[Tpause], they occupy the channel sending the complete IRBECON FRAME. All listening beacons detect the IRBEACON coding, extract the transmitter address (UUID) and store it in their memory unit 5.

3. The cycle is repeated from point 1.

The IR sensors of the transceivers 4 have a threshold [Ee(min)] of IR radiation expressed in [mW/m²]. This threshold indicates that the IR transceiver [Ie] (radiated intensity) must be able to send an IR signal such that upon arrival on the receiver 4, of a second coviTAG 2, the signal Ie>=Ee(min) energy.

If upon arrival on the receiver 4 this signal is equal to Ie<Ee(min) the IR receiver will not be able to discriminate the IR signal sent (Ie) from the ambient IR component which always exists as a component always there of the solar spectrum.

For example but not limited thereto, the infra-red receiver of the coviTAG 2 is the TSOP95656 model produced by Vishay.

The curve illustrated in FIG. 10 shows that the sensitivity of the IR receiver (Ee,min) varies according to the ambient luminosity and switches from 0.1 (in the dark) to over 3 mW/m2 with 8200 lux/m2 which roughly corresponds to the light outdoors in the sun.

The increase of the threshold Ee, as a function of the ambient luminosity, can lead to the blinding of the sensor 14 and therefore to the impossibility of receiving the IR signal.

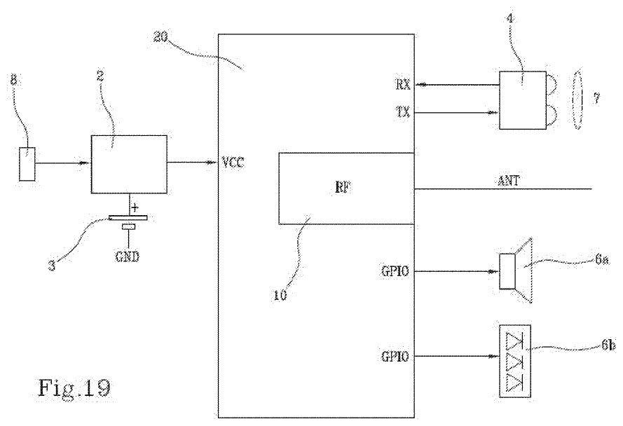

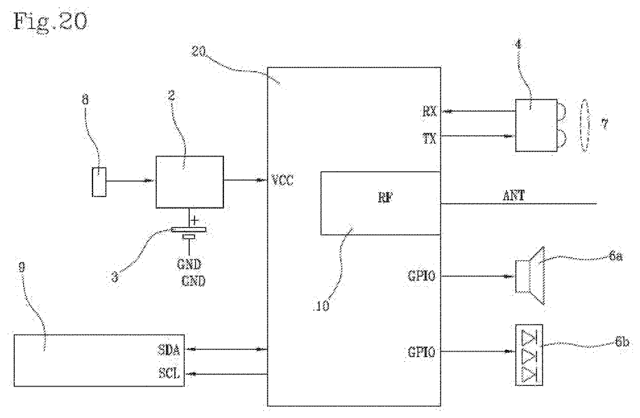

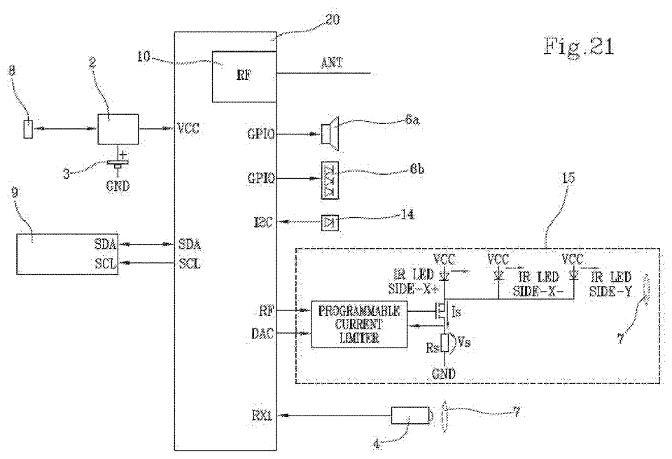

To solve this problem, the present invention contemplates the following solution that includes hardware and communication protocol areas called “Ambient Light Sharing Protocol” (ALSP) and illustrated in FIG. 21. Compared to the architecture illustrated in FIGS. 19 and 20, there are the following two new function blocks:

(9)—Ambient light sensor 14, type OPT3001 Digital ambient light sensor (ALS) with high-precision human-eye response by Texas Instruments or equivalent circuit

(10)—Circuit 15 for programming the current in the IR LED, this circuit 15 is configured to set the desired current value Is through the output of a DAC.

The present invention provides for monitoring the quantity of ambient light through a sensor 14 and for programming the correct current value in the LED 10 as a function of the ambient light. The ambient light value is also included in the IRBEACON communication protocol.

Each coviTag 2 detects the ambient light which hits its sensor 14. When it sends a FRAME FR1, FR2, it enters the light value detected at that moment in a specific data field. All the other beacons that receive the FRAME can thus know that they will have to regulate (increase) the emission energy to reach that beacon.

The information on the amount of light present on each beacon is shared (sharing) with the other beacons, allowing the correct functioning of the data exchange even between coviTAGs 2 subjected to different levels of illumination.

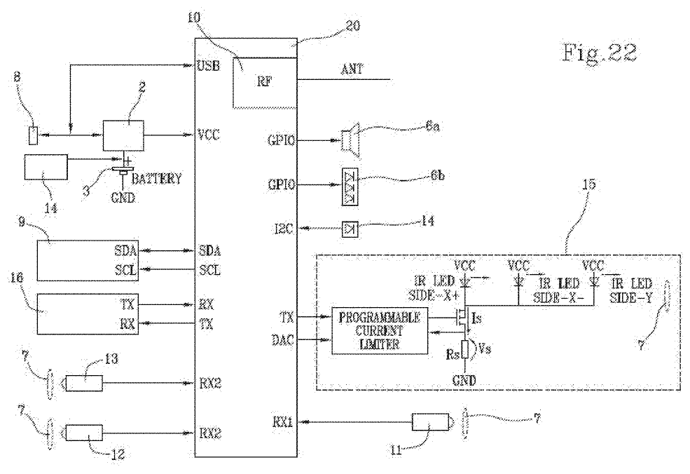

In the preferred embodiment of FIG. 22, the IR receivers present in the transceiver 4 are at least three.

From the technical specifications of the IR receivers currently on the market, such as for example the datasheet of the TSOP59656 receiver by Visahy (www.vishay.com) used, it can be seen that the directivity (and therefore the pick-up angle) is +/−50 degrees referred to the perpendicular of the receiver. Therefore the receiver is able to detect as much as possible what it receives from above and is very penalized laterally (in practice the two radiation patterns receiver and transmitter are orthogonal, which is a problem) because it becomes sensitive and therefore directional on a specific axis.

The use of 2 or 3 IR LED transmitters on one side and as many on the opposite side in order to try and create a sort of transmission bubble assuming to combine the radiation patterns of each single transmitter, unfortunately leads to the creation of the typical array (classic in antennas), with the consequent greater gain of the antenna, in our case to an increase in the radiated power Ie.

To obtain a constructive effect, the dipoles (in our case IR LEDs) must be positioned in a certain way that depends on the wavelength of the IR signal (800-900 nm).

An incorrect positioning would lead to a destructive effect of the IR signal, generating a radiation diagram that is null in some angles. In other words, by placing two or more transmitters (IR LEDs) on the same plane we are creating an array that in the best case would increase the range and reduce the radiation angle (and we would obtain this by working on their relative position that depends on the infra-red wavelength which, being below one micron, becomes practically almost impossible), in order to obtain a bubble we should position the IR transmitter LEDs in a sort of sphere at a distance of at least 10 times the wavelength for the whole space to be covered.

The solution of the present invention, illustrated in FIG. 14, is to position in the infra-red (IR) transceiver 4 a transmitter-receiver pair of IR LEDs 13 on an X+ axis, a transmitter-receiver pair of IR LEDs on an X− axis and a transmitter-receiver pair of IR LEDs on a Y+ axis, perpendicular to the first X axis.

In this way, sooner or later the signals of two coviTAGs 2 will be seen thanks to the simple fact that they are positioned on moving people and not on fixed objects.

In particular, the mechanical solution found for anchoring the IR LEDs overcomes the limit of using LEDs with through hole technology (THT) that offer the advantage of being able to orient the fixing feet with the desired angle but have the limit of being offered on the market with fairly low emission angles, maximum 30°.

Preferably, SMD LED modules that use surface mount technology (SMT) to mount the LED chips on printed circuit boards (PCBs) are used.

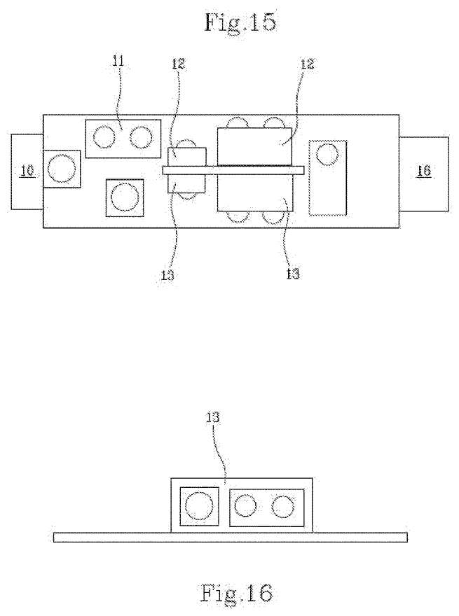

In this way, an emission angle of 60° is obtained, very wide, but being of the SMD LED type it can be positioned on one axis only, the present invention provides for the use of two printed circuits (PCBs), illustrated in FIGS. 15 and 16.

PCB A of FIG. 15 houses the pair of receiver/SMD LEDs oriented in the Y direction (11), while PCB B of FIG. 16 houses the two pairs of receivers placed on the X+ and X− axis.

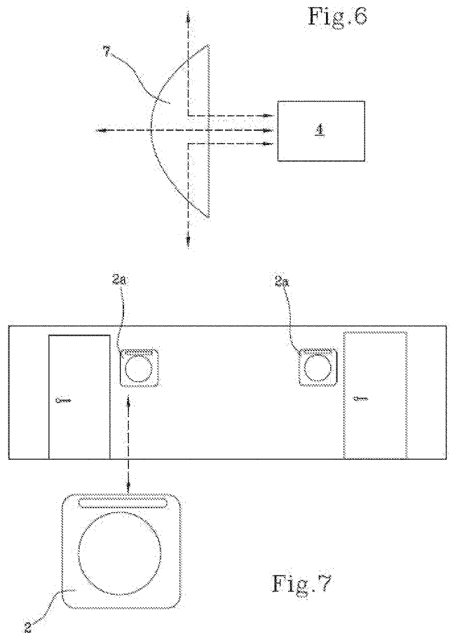

Advantageously, the infra-red transceiver 4 comprises a Fresnel lens 7 (the operation of which is shown in FIG. 6).

Purely indicative, the operation of a Fresnel lens is reported, in this case it is a commercial product made by MURATA (www.murata.com/en-eu).

The advantage of the Fresnel lens lies in the possibility of creating a solid emission angle (volumetric emission) by saturating a specific volume measurable in steradians.

In another preferred embodiment, the ambient light sensor 14 is accompanied by a motion sensor configured to recognize the presence of a target, such as the fingers of the hand or the whole hand.

The presence of the motion sensor allows the user the ability to send commands to the device 2, such as an alarm silence command or other inputs.

This contactless input system allows the coviTAG 2 to be housed in a casing with the highest level of environmental protection up to IP68.

By way of non-limiting example, a motion sensor “Fully Integrated Proximity and Ambient Light Sensor With Infrared Emitter, I2C Interface, and Interrupt Function VCNL4020” by Vishay can be used, which also natively has a luminosity sensor 14.

In another preferred embodiment, the electronic device 2 comprises, in addition to the infra-red transceiver 4, a second radio frequency transceiver, configured to transmit and receive wireless radio frequency (RF) signals.

In this way, it is possible to scan for the presence of other coviTAGs 2 present in the action radius r of the device and the simultaneous activation of the ADVERTISING beacon BLE function. The RF channel can also be a technology other than Bluetooth, it can for example use a subGHz transceiver to improve detection distance and further reduce power consumption of the battery 3. The Bluetooth module is indicated in the attached figures with the reference number 10.6

The radio frequency (RF) signal can be a Bluetooth signal type BLE or RF subGHz or ZigBee RF4CE or UWB.

The electronic devices 2, in order to be able to locate and/or communicate with each other, can therefore use two different physical transmission means (IR or RF/BLE). The use of the IR or RF/BLE channel can be decided according to the environment in which the coviTAGs 2 will be used and according to production costs.

A proprietary protocol will be implemented on the IR channel, described below, which will replicate the operation of the BLE radio beacons on the IR channel.

UWB-IR Hybrid System

The current technology available allows the use of components (modules) called Ultra Wide Band for determining the distance between two objects, their operating principle is similar to that of the radar. It uses radio waves to determine the position of one module with respect to another.

The UWB transceiving modules are adapted to determine the distance in a very precise way, however they have the disadvantage that, like all radio technologies, they can overcome obstacles such as walls or partitions present in offices.

This capability, very useful in the context of radio telecommunications, is here a disadvantage because it would result in false positives, i.e. it would also detect TAGs 2 placed in other compartments or rooms, separated by walls, invalidating the purpose of this invention which is to identify the physical contact between people in line of sight (without separation through walls, for example).

To overcome this problem, we have developed a UWB-IR architecture defined by us as hybrid.

In this way, the advantage of the UWB system of determining the presence of other devices 2 is combined with the infra-red ability to couple TAGs 2 only if they are in line of sight.

In other words, every time the UWB channel is coupled with one or more TAGs 2, to declare the effective coupling and avoid false positives, the system also queries the IR channel and only if it also receives the IR coupling code from the IR channel, only in that moment, TAG 2 is considered to be in line of sight and within the threshold, causing the alarm to be activated.

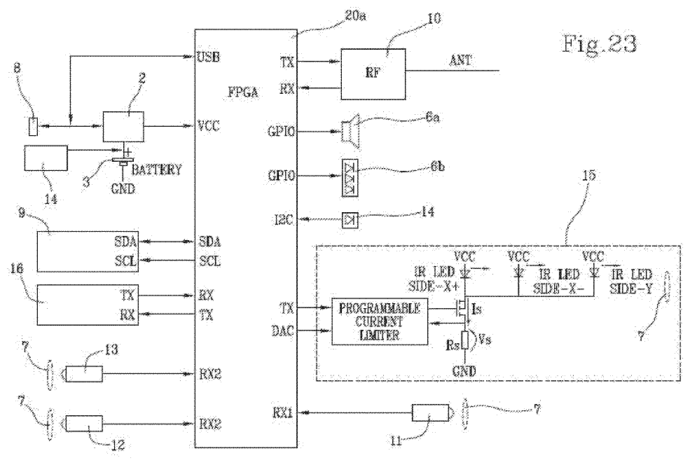

FIGS. 22 and 23 illustrate the block diagram of the hybrid uwb—infra-red system, in which the transceiver module in UWB technology is indicated with 16.

The UWB 16 module is controlled through a communication port which from time to time can be a UART or an I2C or SPI, depending on the module selected in the design step.

The processing unit 20 present in the TAG 2 is further configured to:
search for the presence of an electronic device 2 using the UWB signal of the transceiver;
once an electronic device 2 placed in the action radius (r1) of the UWB transceiver (16) has been coupled, perform the frame (FR1, FR2) exchange using the infra-red signal of the transceivers 4;
generate the alarm signal (S_ALL) if the frame (FR2) transmitted by the second electronic device 2 is received by the IR transceiver 4 of the first electronic device 2.

Preferably, the coviTAG 2 comprises a temperature sensor 9 configured to measure the temperature of the user wearing the coviTAG 2, for example, in the form of a bracelet or smartwatch worn on the wrist or integrated into the facial mask 60.

Multi Target Infra-Red Radar (RIMT)

The use of radio waves to determine the distance between two electronic devices 2 within environments where people work, such as hospitals, offices and homes, may be impractical or may be prohibited by regulations, for the protection of health of people or because they could interfere with electro-medical machinery.

The use of radio waves as a radar source to search for short range targets may be even more delicate. In this case, the possibility of installing, inside a building, dozens and dozens of radar antennas that continuously irradiate the people present might cause alarm.

The use of a radar system to determine the distance between people is very complex and expensive to implement because it requires a very complex system to process the echoes returning from any obstacle present in the radar's action radius, such as furniture, chairs, and other objects present in the various environments and reflective surfaces, a high workload (computation capacity) is required to be assigned to the micro controller.

The present invention provides in a preferred embodiment, illustrated in the block diagram of FIG. 23, the presence of an infra-red radar capable of measuring the distance between two coviTAGs 2.

This solution uses infra-red (light) pulses of negligible power, preferably with a frequency comprised between 20-100 KHz.

The infra-red pulse is outside of any restriction due to specific standards and is particularly inexpensive to realise compared to a classic radio frequency radar.

In this embodiment, the processing unit 20 also comprises internally a calculation module configured to calculate the distance d between two coviTAGs 2 using the infra-red signals of the transceiver 4.

With respect to FIG. 22, this embodiment according to the present invention provides for the replacement of the microprocessor which constitutes the processing unit 20, with a “Field-Programmable Gate Array” (FPGA) device which contains inside it a processor and a distance calculation block RIMT.

The FPGA module of the processing unit 20 comprises a RIMT block or module responsible for calculating the distance between two coviTAGs 2.

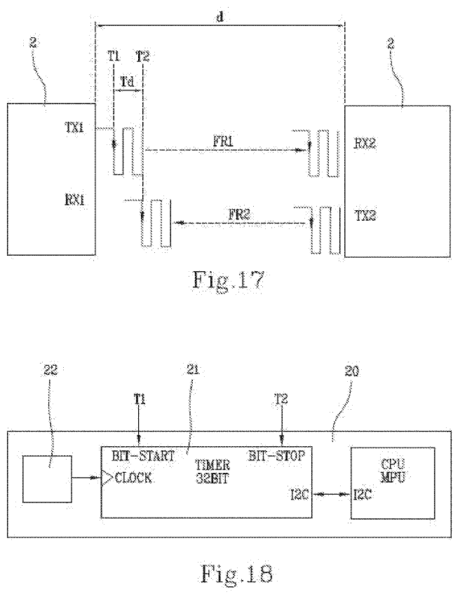

FIG. 17 schematically illustrates the operation of the RIMT calculation module of two coviTAGs 2 with infra-red IR transceiver 4.

A first coviTAG 2 sends to the instant [T1] a first infra-red frame FR1 containing its UUID1 (address).

The first transmitted frame FR1 is received by a second coviTAG 2 and processed. The second coviTAG 2 sends a second frame FR2 containing UUID1 (of the first coviTAG) in addition to the UUID2 (its own covitag 2). Inside the FPGA of the processing unit 20 there is a timer powered by a clock frequency of at least 400 MHz (period=2.5 nS). This timer is triggered on the first falling edge of the emitted signal (T1) and stopped on the arrival of the second FRAM2 (T2). The difference between the two times T2 and T1 (Td=T2−T1) represents the time elapsed to receive and transmit the two FR1+FR.

The time Td is calculated taking into account the following times:
1. Tframe1 (time taken for the first packet FR1 to arrive from the transmitter of the first coviTAG to the receiver of the second covitag), this time is known;
2. Tdelay-rx2 (latency time of the infra-red receiver) this time is known;
3. TCPU2 (processing time, interpretation of FR1 by the second coviTAG 2) known time;
4. Tframe2=Tframe1 (duration of the second packet FR2), this time is known;
5. Tdelay-rx1 (latency time of the infra-red receiver) this time is known;
6. TCPU1 (processing time, interpretation of FR1 by coviTAG 2) known time;
7. Tframe1 (duration of the packet FRAM2) this time is known;
8. Tdistance: Propagation time of the infra-red signal (speed of light=300M meters/sec.), variable to be determined.

Then, the processing unit 20 will calculate the variable Tdistance as follows:

Tdistance=Td−Tframe1−Tdealy-rx2−TCPU2−Tframe2−Tdealy-rx1−TCPU2.

Distance [d] (metres) between the two coviTAGs=300M/Tdistance=>metres

Since the calculation of the distance is activated only if the returned UUID is contained in the FR2, the infra-red return echoes generated by any bounces of FRAME1 on any obstacles encountered by the signal are immediately filtered.

FIG. 18 illustrates a block diagram of an embodiment of the RIMT computing module of the processing unit 20.

The 32-bit timer 21 is initially reset by the CPU via I2C and arranged to be activated on the falling edge of the T1 signal obtained by sending the first packet FR1. 22 indicates an oscillator of at least 400 Mhz. Once the start on T1 has been received, the timer 21 starts counting the time with a resolution, for example, equal to 2.5 nS if a 400 MHz clock is used.

In order to travel two (2) metres the light takes Tdistance(metres)=2/300*10\hat{ }6 (metres/sec.)=>6.6667 nS. With a 2.5 nS resolution the timer will contain, net of all the aforesaid delays, 6 or 7 bits.

As the clock frequency increases, the accuracy over short distances and the number of counted bits of the timer will increase.

The management of the anti-collision protocol is applicable to the distance calculation system illustrated above as described in the previous chapters on page 16. In this way more than two coviTAGs 2 can work together to determine their own distances from each other.

The block diagram of FIG. 22 shows the architecture to be used to realise a device that discriminates the proximity between two or more devices with the method based on threshold with filter.

While the block diagram of FIG. 23 shows the architecture to be used to discriminate the proximity between two devices with the method for calculating the distance with filter. The architecture of FIG. 23 requires, for its operation, a processing unit 20 in which the CPU of FIG. 22 is replaced by an FPGA which can implement inside it a counter with a clock frequency equal to at least 500 MHz of operation. These high operation frequencies are only achievable with FPGA devices, not with CPUs (low cost).

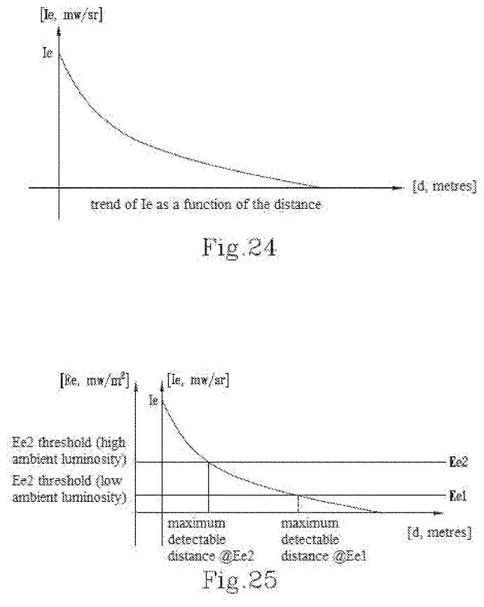

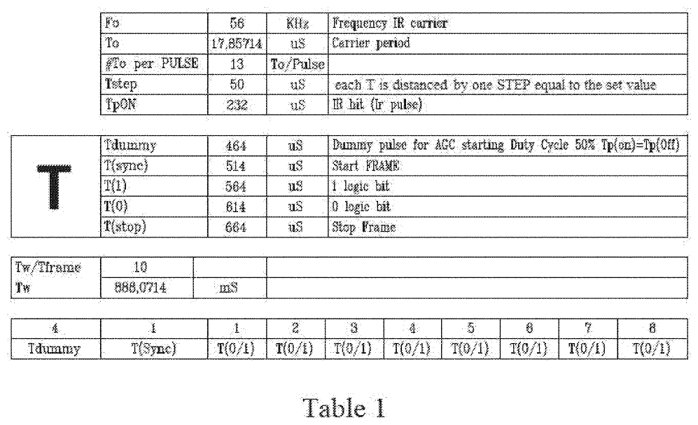

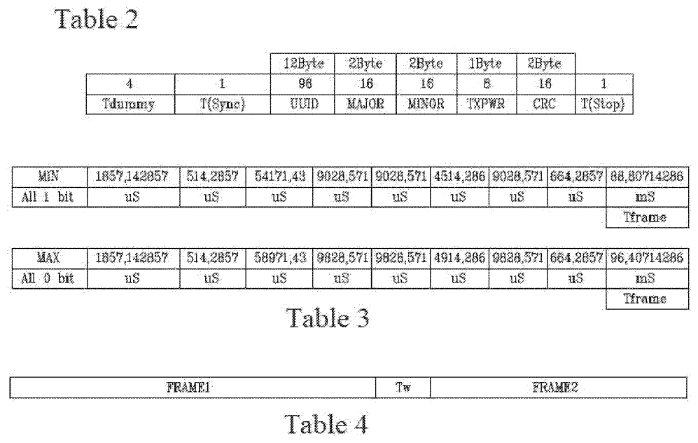

With reference to the curve of FIG. 24, it can be deduced how the energy (radiant intensity) Ie emitted by a generic infra-red LED decreases with the square of the distance. The sensitivity (Ee threshold) that allows the infra-red receiver to discriminate between background noise and signal varies as the ambient luminosity varies is illustrated in FIG. 13.

The maximum distance obtainable from a LED/Receiver pair is calculated with the following formula:

$$d = \sqrt{\frac{Ie}{Ee}} \qquad [ 3 S ]$$

$$\begin{matrix}Ie : Radiant intensity emitted by the infra - red LED \\ Ee : Threshold sensitivity of the infra - red receiver\end{matrix}$$

The superposition of the effects of the threshold of the receiver Ee and of the infra-red signal emitted Ie is illustrated in the curve of FIG. 25, which shows what happens to the distance when the sensitivity (threshold) varies, which in turn depends on the luminosity to which the receiver is subjected.

Therefore—threshold with filter—means that the distance is obtained as a superposition of the two effects of the curve of FIG. 25 but this superposition is a necessary but not sufficient condition, in fact it is necessary that the device also sends a response coded according to the IRBEACON protocol to discriminate (filter) from returns of signals coming from bounces on surfaces (obstacles) and the active detection from parts of other devices (coding).

In the case of the detection system with the method for calculating the distance with filter (FPGA); in this case the FPGA calculates the actual travel time between two FRAME pulses in order to calculate the distance, to avoid being activated on infra-red pulses coming from obstacles (without coding), the return signal must be coded by the second device (filter).

In a third aspect of the invention, a system is provided for tracking movements and respect for the distances between people.

The system 1 comprises a first electronic personal protective device 2 worn by a person as described above and at least one second electronic personal protective device 2 worn by a second person.

The second electronic device 2 is worn by a second person, as schematically illustrated in FIGS. 2 and 3, or it can be fixed near an access point or point of interest to be monitored (FIG. 7).

Preferably, one or more devices 2 of the system 1 can each be associated with the personal electronic communication device (40) (smartphone, smartwatch, tablet, or the like) of the person.

In this case, the processing unit 20 of the electronic device 2 is further configured to associate the electronic personal protective device 2 with the personal electronic communication device (40) of the person wearing it; to use the electronic personal protective device 2 as a radio beacon of a software application present on the electronic personal communication device (40) and to send or receive wireless signals between the electronic device 2 and the personal communication device 40.

The electronic personal communication devices 40 can transmit both a long-distance wireless signal S_ld (for example 2G, 3G, 4G or 5G type mobile radio) to a remote server 50, and a short-distance wireless signal S_sd to the coviTAG 2.

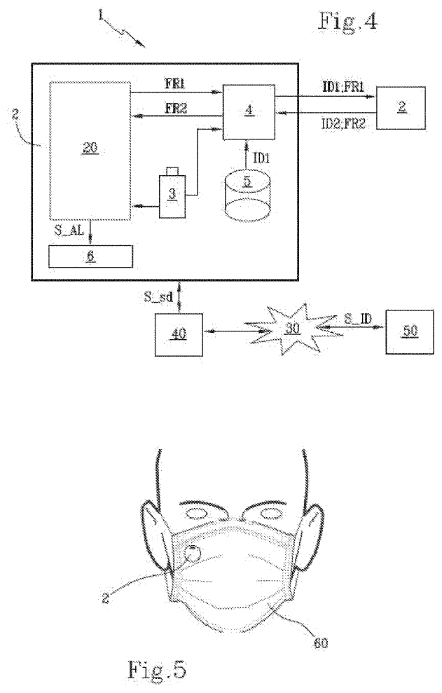

As illustrated in FIG. 4, the electronic devices 2 can also send and receive signals and frames to a remote server 50, using a telematic network 30 to which the personal communication device 40 can be connected.

Operating Procedure

Mode 1: device-device, coviTAGs 2 are worn by people to check distancing.

Mode 2: fixedpoint-device: a coviTAG 2a is positioned in a place, wall, (fixedPoint) as a marker. When a second covidTAG enters the action radius of the fixedpoint, the latter activates and downloads its ID in the coviTAG and at the same time receives the identical ID from the coviTAG and stores it.

In this case, one or more CoviTAGs 2 can be placed at fixed points of interest. As described above, each device has its own unique ID identifying its location.

The CoviTAG 2 positioned in the environment keeps only the IR receiver activated in order to minimise consumption. This mode can bring the CoviTAG to very low consumption such as to guarantee long-term operation. When the fixed CoviTAG identifies the presence of a coviTAG 2 worn by a moving person, it activates on the BLE or TX IR channel by loading its ID (place visited) into the intercepted device and storing the visitor's ID inside it.

This information can then be downloaded to a host (PC) via BLE for accurate tracking of where and how people have moved afterwards. Alternatively, they can be transmitted to a remote server.

Mode3: smartphone-assistant. In this mode, the coviTAG 2 is paired, via Bluetooth, with the person's smartphone through a configuration procedure. Once the connection is established, the coviTAG 2 will act as a beacon (radio beacon), relieving this function of the smartphone. In this configuration, coviTAG is configured as a system with a radio beacon (BLE Beacon) that informs all those smartphone applications whose purpose is tracking people in order to avoid contagions.

The use of the coviTAG associated with a smartphone has the following technical effects:
allows to put the sw application on one's smartphone in the background, reducing consumption;
improves traceability, because Bluetooth technology on smartphones does not have the same functionalities in the background;
the coviTAG device activates a real RSSI system to understand the distance between people while the same function on smartphones tends to collapse easily if there are too many coviTAGs in the action radius;
in the background, the sw application on the smartphone cannot advertise and has limitations, for example, for the random reading of IDs for privacy reasons. In fact, many APPs currently used must necessarily always be active with the screen always highlighted;
the RSSI realised on smartphones is very critical due to the extreme variability of the characteristic parameters of the various chips installed on the phones. The use of a beacon (coviTAG) makes the RSSI analysis more homogeneous and therefore more reliable;
it overcomes the limitations of geolocation-based sw applications, giving these sw applications greater efficiency, reducing consumption, and providing additional information to the user.

In other words, the use of the coviTAG 2 in combination with a smartphone or tablet allows the creation of a complete system for tracking the person and signalling the optimal distance between people.

CoviTAG connected with a smartphone works on two distinct steps:

Step1: Discovery/Advertising. In this step, coviTAG activates the infra-red receiver to check for the presence of other coviTAGs according to the proprietary IR1 protocol. The purpose of this step is only to determine the presence or absence of other coviTAGs. The use of the infra-red channel allows to keep consumption limited to a minimum, normally an infra-red receiver can consume up to a maximum of a few hundred uAmps against the tens of mA needed to keep an RF receiver always active. In this regard, some examples of datasheets for a normal RF receiver and a standard IrDA IR receiver are shown.

An example of commercial RF receiver, comprises the “TI RF Low power CC2652RB SimpleLink™ 32-bit Arm Cortex-M4F multiprotocol 2.4 GHz wireless MCU with crystal-less BAW resonator” (www.ti.com/product/CC2652RB).

An example of commercial product of IR receiver, comprises the MAXIM MAX3120 Low-Profile 3V, 120 μA IrDA Infrared Transceiver or Vishay's TFDU4101 product which states: Dynamic supply current with Tamb=25° C. VCC1=VCC2=2.4 V to 5.5 V ICC1 40(min), 75(typ)−μA.

Direct comparison between the Texas Instrument's commercial RF receiver and the IR receiver described above: 7.3 mA (RF-RX) versus 75 uA (IR-RX).

Mode4: timer-marker: The device 2 can be integrated into a PPE such as a mask 60 to determine when the expiration is approaching or when the expiry date is exceeded.

In this case the coviTAG 2 is programmed to measure the time passing by with its own timer. If the timer exceeds the pre-defined thresholds, it warns the user that the PPE it is associated with is about to expire. For example, once worn on a mask 60, it can emit a continuous green flash to indicate that the PPE is within the expiry date, it can change colour turning to yellow, when it approaches the expiry date and change the colour of the LED to red to indicate that the expiry date has been exceeded. The timer starts with the removal of an insulating tab which isolates the battery 3 from the circuit. The removal is made by pulling, when the pull tab is removed the positive pole of the battery is put in contact with the electronic circuit which at this point by activating can start counting the time passing by.

Mode5 thermo-alarm: In this case the coviTAG 2 will be made in the form of a bracelet or band to be worn on the arm or leg, which, in addition to all the operations provided for in point mode1, device-device, will be equipped with a temperature sensor 9 like the commercial type by Maxim (www.maximintegrated.com), model MAX30208. In this case the block diagram is the one illustrated in FIG. 20.

In a fourth aspect of the invention, a safety system is provided for tracking movements and respect for the distances between people, comprising the steps of:
a) providing an electronic device 2 worn by a person;
b) sending and receiving wireless signals (FR1, FR2) with predetermined power and sensitivity as a function of the action radius (r) which is to be monitored through a transceiver 4;
c) generating and sending to said transceiver 4 a frame (FR1, FR2) at programmable time intervals (Tadv);
d) receiving and processing frames (FR1, FR2) sent from other electronic personal protective devices 2 placed within said action radius (r);
e) generating an alarm signal (S_AL), if a second electronic device 2 is located within said action radius (r);
f) sending said alarm signal (S_AL) to an actuator (6).

The method optionally also comprises the steps of:
providing a first and a second electronic device 2 worn by two people;
placing each electronic device 2 so as to listen to the shared transmission channel (IR);
if they do not detect the start of other transmissions, transmitting a first frame (FR1);
if the frame received (FR2) is different from the first frame transmitted (FR1) at time (Tysnc), detecting a collision:
   immediately interrupting the transmission of frames (FR1, FR2);
   they are paused for a time Tpause.

The pause time is calculated by a special module of the processing unit 20, as a number comprised between 0 and 2\hat{ }(K−1)*T, where T is the time of transmission of the message and [K] is a coefficient that takes into account the number of collisions that have already occurred.

The coviTAGs that detect the collision resume listening on the IR channel, always waiting at least for a time [Tslot] calculated starting from the time [Tsync]+[Tpause] they occupy the channel sending a complete IRBECON FRAME.

All listening beacons detect the IRBEACON coding, extract the transmitter address (UUID) and store it in their buffered memory unit 5.

In a fifth aspect of the invention, a method as described above is provided, in which one or more steps are carried out by means of a computer.

What has been described up to now for the sake of clarity and convenience in relation to two devices CoviTAG 2 worn by two people, is intended to be extended to a greater number of people involved, each wearing a tag.

As a person skilled in the art can easily understand, the invention allows overcoming the drawbacks highlighted above with reference to the prior art.

In particular, the present invention makes it possible to improve the safety of people, in any environment in which there may be gatherings, such as for example a work environment, a point of sale or in the public means of transport or in any environment at risk of potential approaches between people beyond certain risk distances.

It is clear that the specific features are described in relation to different embodiments of the invention with an exemplary and non-limiting intent. Obviously a person skilled in the art can make further modifications and variants to the present invention, in order to satisfy contingent and specific needs. For example, the technical features described in relation to an embodiment of the invention can be extrapolated therefrom and applied to other embodiments of the invention. Such modifications and variations are moreover embraced within the scope of the invention as defined by the following claims.

1. An electronic personal protective device (2) comprising:
a rechargeable battery (3);
a transceiver (4) configured to send and receive wireless signals (FR1, FR2) with predetermined power and sensitivity as a function of the action radius (r) which is to be monitored;
a memory unit (5) containing a unique recognition code (ID1, ID2);
an actuator (6) configured to warn the person wearing said electronic device (2);
a processing unit (20) configured to:
generate and send to said transceiver (4) a frame (FR1, FR2) at programmable time intervals (Tadv);
receive and process frames (FR1, FR2) sent from other electronic personal protective devices (2) placed within said action radius (r);
generate an alarm signal (S_AL), if a second electronic device (2) is located within said action radius (r);
send said alarm signal (S_AL) to said actuator (6).

2. The electronic personal protective device (2) according to claim 1, wherein said transceiver (4) is an infra-red (IR) transceiver (4), configured to transmit and receive said wireless signals in the infra-red spectrum.

3. The electronic personal protective device (2) according to claim 2, wherein said infra-red (IR) transceiver (4) comprises:
a transmitter-receiver pair of IR LEDs, on a X+ axis;
a transmitter-receiver pair of IR LEDs, on a X− axis;
a transmitter-receiver pair of IR LEDs, on a Y+ axis, perpendicular to the first X axis;

4. The electronic personal protective device (2) according to claim 2 or 3, wherein the transceiver (4) comprises a Fresnel lens (7).

5. The electronic personal protective device (2) according to one or more of the preceding claims, wherein said transceiver (4) transmits and receives said wireless signals in radio frequency (RF).

6. The electronic personal protective device (2) according to claim 5, wherein said radio frequency (RF) signal is a Bluetooth signal such as BLE or RF subGHz or ZigBee RF4CE or UWB.

7. The electronic personal protective device (2) according to claim 5, wherein said processing unit (20) is further configured to:
search for the presence of an electronic device (2) using the UWB signal of the transceiver (16); and
once an electronic device (2) placed in the action radius (r1) of the UWB transceiver (16) has been coupled, perform the frame (FR1, FR2) exchange using the infra-red signal of the transceivers (4);
generate the alarm signal (S_ALL) if the frame (FR2) transmitted by the second electronic device (2), is received by the transceiver IR (4-1) of the first electronic device (2).

8. The electronic personal protective device (2) according to one or more of the preceding claims, wherein said actuator (6) is a buzzer (6a) and/or a high luminosity RGB LED (6b).

9. The electronic personal protective device (2) according to one or more of the preceding claims, comprising a temperature sensor (9) configured to measure the temperature of the user wearing the electronic device (2).

10. The electronic personal protective device (2) according to one or more of the preceding claims, wherein the electronic device (2) comprises a box-shaped casing that can be worn by a person and conformed to be worn as:
a button;
a pin;
a bracelet;
an armband;
a pendant to apply to a necklace;
a badge strap;
a belt.

11. The electronic personal protective device (2) according to one or more of the preceding claims, wherein the electronic device (2) comprises:
an environmental luminosity sensor (14); and
a circuit (15) for the regulation of current in the infra-red transceiver (4) IR LED configured to set the desired current value (Is) through the output of a DAC as a function of the environmental luminosity.

12. The electronic personal protective device (2) according to claim 11, wherein said processing unit (20) is configured to:
insert, in a relevant field present in the frame (FR1, FR2), the environmental luminosity value generated at a certain moment by said environmental luminosity sensor (14).

13. The electronic personal protective device (2) according to claim 11 or 12, comprising a movement detection sensor configured to recognise the approach of the fingers of a hand or the hand of a person for sending commands to the electronic device (2).

14. A personal protective device, in particular a facial protection mask (60), comprising an electronic device (2) according to one or more of the preceding claims.

15. The personal protective device according to claim 14, wherein the electronic device (2) comprises a timer configured to send a signal to the actuator (6) able to warn the user wearing the personal protective device of the approach of the expiry date.

16. A safety system (1) for tracking movements and respect for distances between people, comprising:
a first electronic personal protective device (2) worn by a person according to one or more of the preceding claims;
a second electronic personal protective device (2).

17. The safety system (1) according to claim 16, wherein the second electronic device (2) is worn by a second person or is fixed in proximity to an access point to be monitored.

18. The safety system (1) according to claim 16 or 17, comprising an electronic personal communication device (40) that can be associated with the electronic device (2) of the person and wherein the processing unit (20) of the electronic device (2) is further configured to:
associate the first electronic personal protective device (2) with the electronic personal communication device (40) of the person wearing it;
use the electronic personal protective device (2) as the radio beacon of a software application present on the electronic personal communication device (40).

19. A safety method for tracking movements and respect for distances between people, comprising the steps of:
providing an electronic device (2) worn by a person;
sending and receiving wireless signals (FR1, FR2) with predetermined power and sensitivity as a function of the action radius (r) which is to be monitored through a transceiver (4);
generating and sending to said transceiver (4) a frame (FR1, FR2) at programmable time intervals (Tadv);
receiving and processing frames (FR1, FR2) sent from other electronic personal protective devices (2) placed within said action radius (r);
generating an alarm signal (S_AL), if a second electronic device (2) is located within said action radius (r);
sending said alarm signal (S_AL) to an actuator (6).

20. The method according to claim 19, comprising the steps of:
providing a first and a second electronic device (2) worn by two people;
placing each electronic device (2) so as to listen to the shared transmission channel (IR);
if they do not detect the start of other transmissions, transmitting a first frame (FR1);
if the frame received (FR2) is different from the first frame transmitted (FR1) at time (Tysnc), detecting a collision:
immediately interrupting the transmission of frames (FR1, FR2);
they are paused for a time (Tpause).

21. The method according to claim 19 or 20, wherein one or more steps are implemented by computer.
